(19) United States
(12) Patent Application Publication
(10) Pub. No.: US 2010/0056902 A1
(43) Pub. Date: Mar. 4, 2010

(54) PATIENT POSITIONING COUCH AND MEDICAL DEVICE WITH A PATIENT POSITIONING COUCH

(76) Inventors: Alexander Granzer, Lauf A.D. Pegnitz (DE); Bernd Maciejewski, Markt Erlbach (DE); Jürgen Nistler, Erlangen (DE); Wolfgang Renz, Erlangen (DE); Lothar Schön, Neunkirchen (DE); Günther Zebelein, Mohrendorf (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/546,013

(22) Filed: Aug. 24, 2009

(30) Foreign Application Priority Data
Aug. 27, 2008 (DE) 10 2008 044 643.2

(51) Int. Cl. A61B 5/05; A61G 99/00; G01R 33/44

(52) U.S. Cl. 600/415; 128/845; 324/307

(57) ABSTRACT

A patient positioning couch includes an illumination apparatus having at least one light source that is arranged in the patient positioning couch. The patient positioning couch is capable of being placed in an examination area of a medical device. Through the arrangement of the at least one light source of the illumination apparatus of the patient positioning couch, it is always ensured that the illumination does not lose its location relative to a patient or another object positioned on the patient positioning couch, even if the position of the patient positioning couch changes, e.g. if the patient positioning couch is moved into or out of the examination area. At the same time an especially space-saving illumination of the examination area can thus be achieved.

### CROSS REFERENCE TO RELATED APPLICATIONS

This application claims priority of German application No. 10 2008 044 643.2 filed Aug. 27, 2008, which is incorporated by reference herein in its entirety.

### FIELD OF THE INVENTION

The invention relates to a patient positioning couch and a magnetic resonance device with a patient positioning couch.

### BACKGROUND OF THE INVENTION

In an examination of a patient by means of a magnetic resonance device the patient is positioned on a patient positioning couch and brought together therewith into an examination area of the magnetic resonance device. Such examination areas are usually realized like a cylinder and extend like a tunnel through a magnet unit of the magnetic resonance device. The examination area typically has a diameter that is only slightly larger than the average shoulder width (approx. 60 cm) of a patient.

Within the tubular examination area of the magnetic resonance device there is therefore little space between the patient and a wall that delimits the examination area from the magnet unit of the magnetic resonance device. In order to reduce feelings of anxiety in patients in this examination situation, which is often felt to be restrictive, illumination systems are known in which light sources are affixed in or to a trim panel of the examination area, and which shed light into the examination area for example from an outer edge of an opening of the examination area. However an optimal illumination of the examination area that has a calming effect on a patient can rarely be achieved, because the patient himself or other units of the medical device located within the patient tunnel cast shadows, for example.

Better illumination is achieved with an illumination apparatus arranged within the examination area. However such apparatuses demand a considerable amount of the already tight space in the examination area and/or require a larger examination area, which is costly. An example of such an illumination system is described in DE 10 2004 024 095 A1.

### SUMMARY OF THE INVENTION

It is an object of the invention to specify a patient positioning couch and a magnetic resonance device that increase a patient's comfort during a magnetic resonance examination.

The objects are achieved in accordance with the invention by a patient positioning couch and a medical device as claimed in the claims.

Accordingly a patient positioning couch according to the invention comprises an illumination apparatus having at least one light source that is arranged in the patient positioning couch. The patient positioning couch is capable of being placed in an examination area of a medical device. Through the inventive arrangement of the at least one light source of the illumination apparatus of the patient positioning couch, it is always ensured that the illumination does not lose its location relative to a patient or another object positioned on the patient positioning couch, even if the position of the patient positioning couch changes, e.g. if the patient positioning couch is moved into or out of the examination area. At the same time an especially space-saving illumination of the examination area can thus be achieved.

A medical device according to the invention comprises an examination area and a patient positioning couch with an illumination apparatus comprising at least one light source that is arranged in the patient positioning couch, the patient positioning couch being capable of being placed in the examination area.

The advantages referred to in relation to the patient positioning couch apply analogously.

### BRIEF DESCRIPTION OF THE DRAWINGS

Further advantages and details of the present invention will emerge from the exemplary embodiments described below and with the aid of the drawings. Examples are provided below, which do not constitute any limitation of the invention, and in which:

FIG. 1 shows a schematic plan view of a section of a patient positioning couch according to the invention,

FIG. 2 shows a schematic frontal view of an embodiment of the patient positioning couch within an examination area of a magnetic resonance device,

FIG. 3 shows a schematic sectional view of a magnetic resonance device with a patient positioning couch.

### DETAILED DESCRIPTION OF THE INVENTION

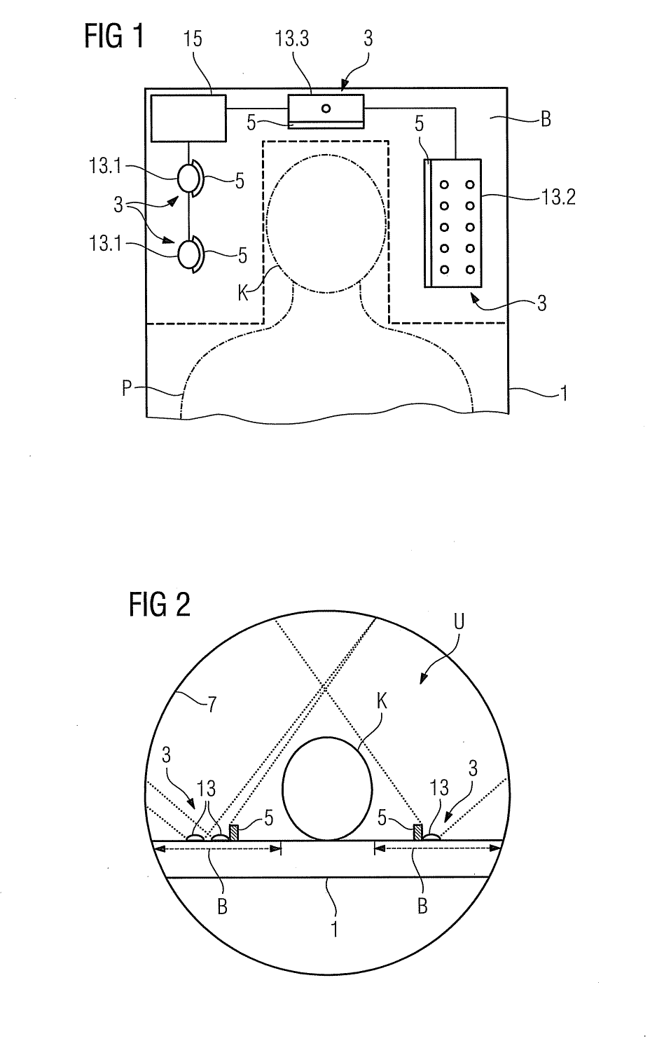

FIG. 1 schematically shows the basic design of a patient positioning couch 1 according to the invention, with just one section of the patient positioning couch 1 being shown.

When a patient P is positioned on the patient positioning couch 1, the head K of the patient P comes to rest in the vicinity of the head end of the patient positioning couch 1 at a position intended for the head K, as illustrated in FIG. 1 with the aid of the patient P shown with dot-dashed lines. This position that is intended for the head K is located approximately at the center of the head end of the patient positioning couch 1. Adjacent to the position of the head K, further delimited by the edges/boundaries of the patient positioning couch 1 and by approximation by the position of the shoulders of the patient P, a further area B is located at the head end of the patient positioning couch 1, which is indicated in FIG. 1 by the dashed lines.

The patient positioning couch 1 comprises an illumination apparatus 3 with at least one light source 13.1, 13.2, 13.3 advantageously arranged in the patient positioning couch 1 in the area B just described adjacent to the position provided for the head K. Here the illumination apparatus 3, with the at least one light source 13.1, 13.2, 13.3, is arranged laterally adjacent to the left and/or right side of the position provided for the head K and/or in the direction of the body axis of the patient P above the position provided for the head K.

Through the arrangement of the at least one light source 13.1, 13.2, 13.3 of the illumination apparatus 3 in the area B adjacent to the position provided for a head K of a patient P on the patient positioning couch 1 it is easily possible to prevent the at least one light source 13.1, 13.2, 13.2 of the illumination apparatus 3 becoming covered by arms, legs or other body parts of a patient P located on the patient positioning couch 1. This therefore ensures that the illumination apparatus 3 can always provide sufficient brightness in the area of the head K of a patient P located on the patient positioning couch 1 such that the brightness, which can be perceived by the patient P, calms him and thus increases his comfort.

In order to illustrate various possibilities, FIG. 1 shows three variants of possible light sources 13.1, 13.2, 13.3. In fact the illumination apparatus 3 can also comprise just one light source 13.1, 13.2, 13.3 or another combination of light sources 13.1, 13.2, 13.3 that differs from the combination shown. Likewise the light sources 13.1, 13.2, 13.3 can also be arranged in another way, but preferably still in the area B.

A light source 13.1 is recessed for example in the patient positioning couch 1 and/or protrudes only a small amount from the patient positioning couch 1, and radiates a cone of light upward. By combining various such light sources 13.1 a variable illumination situation can be created by overlaying the various cones of light produced, or possibly also by using different colored light in the light sources 13.1.

As an alternative, or in combination therewith, a light source 13.2 can be used that itself takes up a larger space, e.g. with a width of a few centimeters and a length of up to several decimeters, on the patient positioning couch 1. This light source 13.2 is preferably also recessed in the patient positioning couch 1 or is only raised a few centimeters from the patient positioning couch 1. A light source 13.2 of this type generates a large area of illumination. The light source 13.2 here can comprise e.g. several or different colored LEDs (light emitting diodes).

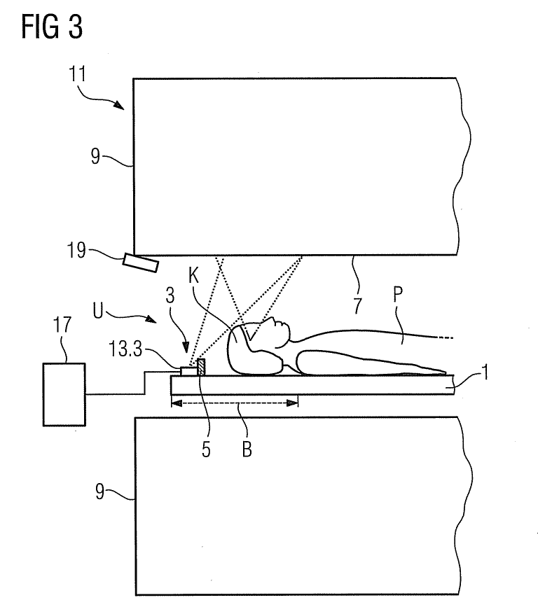

As a further alternative, or in combination therewith, a light source 13.3 can be used that comprises e.g. at least one laser. With the aid of a light source 13.3 of this type, pictures and/or information for example can be projected onto a surface surrounding the patient positioning couch 1. More details are provided with reference to FIG. 3.

The light sources 13.1, 13.2, 13.2 are each compatible with MR (magnetic resonance), i.e. they do not impinge upon the light source or the interactions that influence an MR examination with magnetic fields and high-frequency fields that occur in particular in an examination area of a magnetic resonance device during an examination.

Advantageously the illumination apparatus 3 further comprises screens 5 that are each arranged between a light source 13.1, 13.2, 13.3 and the head K of a patient P located on the patient positioning couch 1 such that the head K is not directly illuminated by the light sources.

The patient positioning couch 1 further advantageously comprises a plug assembly 15, e.g. for plugging in local coils of a magnetic resonance device or other auxiliary devices that require a power supply for examinations. Thus the cabling and power feed that is already present in the plug assembly 15 can also be used jointly for a power supply to the illumination apparatus 3. Accordingly the power supply to the plug assembly 15 is connected to the illumination apparatus 3. Separate cabling for the illumination apparatus 3 to a power source located outside the patient positioning couch 1 is not necessary.

FIG. 2 shows a frontal view of the patient positioning couch 1. In the position shown the patient positioning couch 1 is located in a cylindrical examination area U of a medical device (not shown here). A surface 7 that extends around the patient positioning couch 1 delimits the examination area U of the medical device as a boundary wall against the medical device. In particular the surface 7 is an interior wall of a patient tunnel of the medical device.

The illumination apparatus 3 arranged in the area B specified in more detail with reference to FIG. 1 is realized such that a direction of illumination of the at least one light source 13 extends in the direction of the surface 7. The surface 7 is reflective so that the light from the light sources 13, which is advantageously strongly scattered, is reflected back into the examination area U. The surface 7 is then illuminated extensively by the illumination apparatus 3 at least in the visual field of a patient positioned on the patient positioning couch. Thus, through the arrangement of the illumination apparatus 3 and the upward direction of illumination of the illumination apparatus 3 toward a surface 7 that delimits the examination area U, a pleasant indirect illumination, which does not dazzle the patient P and can positively influence his mood, is continuously produced for a patient P located on the patient positioning couch 1 in the area of his head, in particular in the visual field of the patient P, in other words where the patient P perceives the illumination. In particular the brightness produced by means of this indirect illumination reduces feelings of constriction that may possibly be experienced by the patient P in the examination area U.

As illustrated by dotted lines, screens 5 for the illumination apparatus 3 are advantageously used to prevent the head K being illuminated by the light sources 13 owing to the arrangement of said screens between the light sources 13 and the head K of a patient P located on the patient positioning couch 1. Dazzling of a patient located on the patient positioning couch 1 by the illumination apparatus 3 is additionally prevented.

Furthermore the dotted lines indicate how different colored light from the light sources 13 can possibly be overlayed and thus mixed on the surface 7 in order e.g. to produce a calming color gradient on the surface 7 that is visible to a patient P located on the patient positioning couch 1.

A section of a medical device 11 is sketched out in FIG. 3 using the example of a magnetic resonance device 11. The magnetic resonance device 11 is represented simply by its magnet unit 9 and further comprises a patient positioning couch 1. Additional units such as control units or high-frequency units are known and for reasons of simplicity are not shown.

The magnet unit 9, which comprises in particular a main magnet and gradient coils, surrounds an examination area U into which a patient P is introduced on a patient positioning couch 1 for an examination. A surface 7 delimits the medical device 11 against the examination area U.

As has already been described in relation to FIG. 1, an illumination apparatus 3 is arranged on the patient positioning couch 1 in an area B adjacent to the position provided for the head K of a patient P located on the patient positioning couch.

In the variant shown here the illumination apparatus comprises just one light source 13.3 that comprises at least one laser, and advantageously one screen 5. The light source 13.3 is connected to an image and/or information source 17. The light source 13.3 is realized so as to project image and/or information data transmitted by the image and/or information source 17 as images and/or information onto the surface 7. Accordingly the images and/or information are projected in an area of the surface 7 that lies in the visual field of a patient P located on the patient positioning couch 1, as indicated by the dotted lines.

The images and/or information to be projected may include for example images that entertain or calm the patient P, e.g. also holographic images, or stimulating images or information linked to fMRT examinations (fMRT: “functional magnetic resonance tomography”), or simply information e.g. about the current status of the examination, such as remaining scan time and the like. If the projection of information and/or images cannot provide sufficient brightness in the examination area U in order to calm the patient P, further light sources such as those described with reference to FIG. 1 can advantageously be used e.g. in order to illuminate areas of the surface 7 laterally adjacent to the position of the head of the patient P and thus to produce sufficient indirect light again.

Through the arrangement of the illumination apparatus 3 on the patient positioning couch 1 and the direction of illumination away from the patient, brightness in the examination area can always be provided at the correct position for the patient P, in the area of his head K, without dazzling the patient P.

This indirect illumination, which is always located level with the head K of the patient P, further allows for easy monitoring of the patient by means of a monitoring apparatus 19, e.g. a camera, or by means of the personnel overseeing the examination, since in particular the face of the patient P is always easily visible by means of the indirect illumination by the illumination apparatus 3.

1.-13. (canceled)

14. A patient positioning couch placed in an examination area of a medical device, comprising:
an illumination apparatus; and
a light source of the illumination apparatus that is arranged in the patient positioning couch.

15. The patient positioning couch as claimed in claim 14, wherein the light source is arranged in an area adjacent to a position provided for a head of a patient on the patient positioning couch.

16. The patient positioning couch as claimed in claim 14, wherein the light source illuminates in a direction that extends in a direction of a reflective surface arranged around the patient positioning couch.

17. The patient positioning couch as claimed in claim 16, wherein the illumination apparatus illuminates the reflective surface extensively in a visual field of a patient positioned on the patient positioning couch.

18. The patient positioning couch as claimed in claim 16, wherein the reflective surface is a wall that delimits the examination area of the medical device.

19. The patient positioning couch as claimed in claim 18, wherein the reflective surface is an interior wall of a patient tunnel.

20. The patient positioning couch as claimed in claim 14, wherein the light source comprises a light emitting diode.

21. The patient positioning couch as claimed in claim 14, wherein the light source comprises a laser.

22. The patient positioning couch as claimed in claim 14,
wherein the light source is connected to an image or information source for receiving an image or information, and
wherein the image or the information is projected by the laser onto a surface in an area of a visual field of a patient located on the patient positioning couch.

23. The patient positioning couch as claimed in claim 14,
wherein the illumination apparatus comprises a screen arranged between the light source and a position of a head of a patient located on the patient couch, and
wherein the screen prevents an illumination of the head by the light source.

24. The patient positioning couch as claimed in claim 14,
wherein the patient positioning couch comprises a plug assembly in an area adjacent to a position provided for a head of a patient, and
wherein a voltage supply to the plug assembly is connected to the illumination apparatus to supply a voltage to the illumination apparatus.

25. A medical device, comprising:
an examination area; and
a patient positioning couch that is placed in the examination area, the patent positioning couch comprising:
an illumination apparatus; and
a light source of the illumination apparatus that is arranged in the patient positioning couch.

26. The medical device as claimed in claim 25, wherein the medical device is a magnetic resonance device.
